(19) United States
(12) Patent Application Publication
(10) Pub. No.: US 2025/0238921 A1
(43) Pub. Date: Jul. 24, 2025

(54) IMAGE PROCESSING SYSTEM AND METHOD FOR ANALYZING OPTIC CUP AND OPTIC DISC

(71) Applicants: Acer Incorporated, New Taipei City (TW); Acer Medical Inc., NEW TAIPEI CITY (TW)

(72) Inventors: Shang-Lin CHUNG, New Taipei City (TW); Chien-Hung LI, New Taipei City (TW); Shin TENG, NEW TAIPEI CITY (TW)

(21) Appl. No.: 18/640,060

(22) Filed: Apr. 19, 2024

(30) Foreign Application Priority Data
Jan. 22, 2024 (TW) 113102398

(51) Int. Cl. G06T 7/00; G06V 10/82

(52) U.S. Cl. CPC G06T 7/0012; G06V 10/82; G06T 2207/20081; G06T 2207/20084; G06T 2207/30041

(57) ABSTRACT

Provided is an image processing system and method for analyzing an optic cup and optic disc. The system and method include the following steps. A processor obtains a fundus image. The processor recognizes the optic disc and optic cup in the fundus image using an image recognition model and marks the outlines of the optic disc and optic cup. An interpretation mode is provided so that interpretation data may be generated for risk assessment according to the outlines of the optic disc and optic cup as marked. The image recognition model is a deep learning model trained by a large amount of pre-collected fundus maps. The outlines of the optic cups and optic discs in the fundus images have been marked in advance.

### CROSS REFERENCE TO RELATED APPLICATIONS

This application claims priority of Taiwan Patent Application No. 113102398, filed on Jan. 22, 2024, the entirety of which is incorporated by reference herein.

### BACKGROUND OF THE INVENTION

### Field of the Invention

The present invention relates to an image processing system and method, and in particular it relates to an image processing system and method for analyzing an optic cup and optic disc.

### Description of the Related Art

Fundus Maps (also known as Fundus Photography) are common medical images that are easy to take. They often serve as first-line diagnostic tools used by ophthalmologists to interpret signs of eye damage for risk assessment. A fundus map has an optic cup and an optic disc included within. The relative size relationship between the optic cup and the optic disc included in the fundus map is an important sign of various eye injuries, such as Glaucoma, Optic Neuritis, Pseudotumor Cerebri, and Leber's hereditary optic atrophy, etc.

However, in the process of risk assessment, since the optic cup is not an anatomical structure with a clear boundary, even if it is marked by an ophthalmologist, it is necessary to consider a variety of factors (such as the color change of the optic cup, the turning change of the optic disc blood vessel, etc.) that affect the outline of the optic cup. To mark the optic cups is therefore much more difficult than with general anatomical structures. Even though a skilled ophthalmologist marks them, it still takes a long time (for example, more than three minutes) to evaluate and actually mark. In addition, when a fundus map is obtained from an object with high myopia, it is more difficult to recognize the edge of the optic cup than that of another object without high myopia. This makes it more difficult for ophthalmologists to interpret the image, and the time required to mark the optic cup and the optic disc is increased.

Therefore, for the interpretation of fundus maps, adding an auxiliary function for marking the outlines of the optic cup and the optic disc is indeed expected to improve the quality of interpretation, and at the same time, it can reduce the time spent by ophthalmologists on marking and interpretation assessment.

### BRIEF SUMMARY OF THE INVENTION

An embodiment of the present invention provides an image processing system for analyzing an optic cup and optic disc. The image processing system includes a processor that accesses a program to perform the following operations. The processor receives a fundus map, and uses an image recognition model to recognize an optic cup and an optic disc in the fundus map, to mark the outlines of the optic cup and the optic disc. In an interpretation mode, the processor generates interpretation data for risk assessment of the eyes, according to at least the outlines of the optic cup and the optic disc. The image recognition model is a deep learning model, which has been trained by using pre-collected fundus maps as training data, and on each of the pre-collected fundus maps, and the outlines of the optic cup and the optic disc have been previously marked by an ophthalmologist.

According to one embodiment of the present invention, the image processing system for analyzing the optic cup and optic disc further includes a display device and an input device. In a marking mode, the processor further performs operations to mark the outlines of the optic cup and the optic disc with a plurality of outline points, to display the plurality of outline points on the display device, and through the input device, to receive instructions to modify, clear, or accept the plurality of outline points, from the ophthalmologist.

In addition, an embodiment of the present invention provides an image processing method for analyzing an optic cup and optic disc, applied to an electronic apparatus. The method includes the steps performed by a processor of the electronic apparatus. The steps include recognizing an optic cup and an optic disc in the fundus map, and marking outlines of the optic cup and the optic disc; in an interpretation mode, generating interpretation data for risk assessment of eyes according to at least the outlines of the optic cup and the optic disc. The image recognition model is a deep learning model, which has been trained by using pre-collected fundus maps as training data, and on each of the pre-collected fundus maps the outlines of the optic cup and the optic disc have been previously marked by an ophthalmologist.

### BRIEF DESCRIPTION OF THE DRAWINGS

The present invention can be more fully understood by reading the subsequent detailed description and examples with references made to the accompanying drawings, wherein:

FIG. 1 is a block diagram showing an image processing system for analyzing an optic cup and an optic disc, according some exemplary embodiment of the present invention.

FIG. 2 shows a flow chart of an image processing method for analyzing an optic cup and an optic disc, according to some exemplary embodiment of the present invention.

FIG. 3 shows an original fundus map without marking, and a fundus map having been marking its optic disc and optic cup.

### DETAILED DESCRIPTION OF THE INVENTION

The following description is of the best-contemplated mode of carrying out the invention, and is made for illustrating the general principles of the invention and should not be taken in a limiting sense. The scope of the invention is best determined by reference to the appended claims.

It will be further understood that the terms “comprises,” “comprising,” “includes” and/or “including,” when used herein, specify the presence of stated features, numeral values, steps, operations, elements, and/or components, but do not preclude the presence or addition of one or more other features, numeral values, steps, operations, elements, components, and/or groups thereof. Use of ordinal terms such as “first”, “second”, “third”, etc., in the claims to modify a claim element, does not connote any priority, precedence, or order of one claim element over another or the temporal order in which acts of a method are performed, but are used merely to distinguish one claim element having a certain name from another element having the same name.

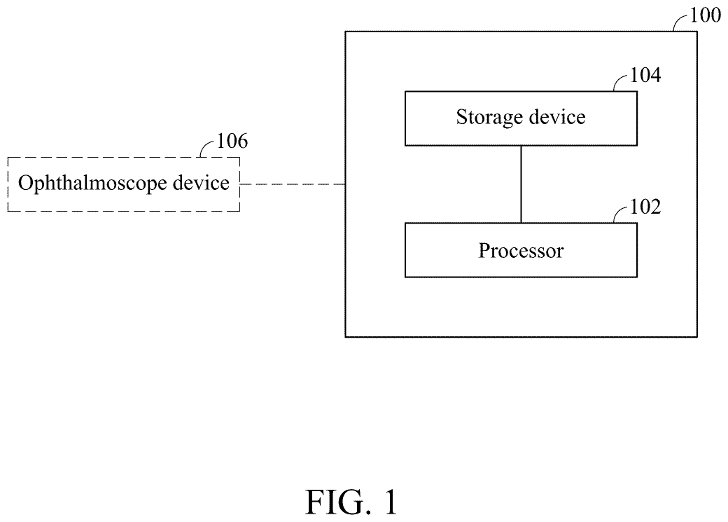

FIG. 1 is a block diagram showing an image processing system 100 for analyzing an optic cup and an optic disc, according some exemplary embodiment of the present invention. In some embodiment, the image processing system 100, for example is an electronic device of various computer devices and/or smart devices, but is not limited thereto. In this embodiment, the image processing system 100, for example, is a notebook computer.

In FIG. 1, the image processing system 100 includes a processor 102, and a storage device 104. The storage device 104, for example, is any type of Random Access Memory (RAM), Read-Only Memory (ROM), Flash Memory, Hard Disk or other similar devices, or a combination of the above devices, and can be used to store multiple programs, models or modules.

The processor 102 is coupled to the storage device 104, and may be a general purpose processor, special purpose processor, traditional processor, digital signal processor, multiple microprocessors, one or more microprocessors combined with a digital signal processor core, controller, microcontroller, ASIC (Application Specific Integrated Circuit), FPGA (Field Programmable Gate Array), any other kind of integrated circuit, state machine, ARM (Advanced RISC Machine) processors, and similar electronic products.

Referring to FIG. 1, the image processing system 100 obtains a fundus map of a detection object for image processing and analyzing, through an external ophthalmoscope device 106.

In one embodiment, the ophthalmoscope device 106 can be a direct ophthalmoscope, or an indirect ophthalmoscope. Taking the direct ophthalmoscope as an example, the ophthalmoscope device 106 can directly inspect the fundus without dilating pupils, thus no need to perform examination in a dark room.

In one embodiment, the ophthalmoscope device 106 is a digital fundus camera, which generally uses a digital camera with more than 2 million pixels to capture high-definition fundus maps. The digital camera is connected to a dedicated interface of the fundus camera, captures the required fundus maps, and then transmits the fundus maps to the image processing system 100 for image analysis, storage, and printing, etc. In one embodiment, the processor 102 is configured to receive the fundus image and perform image analysis.

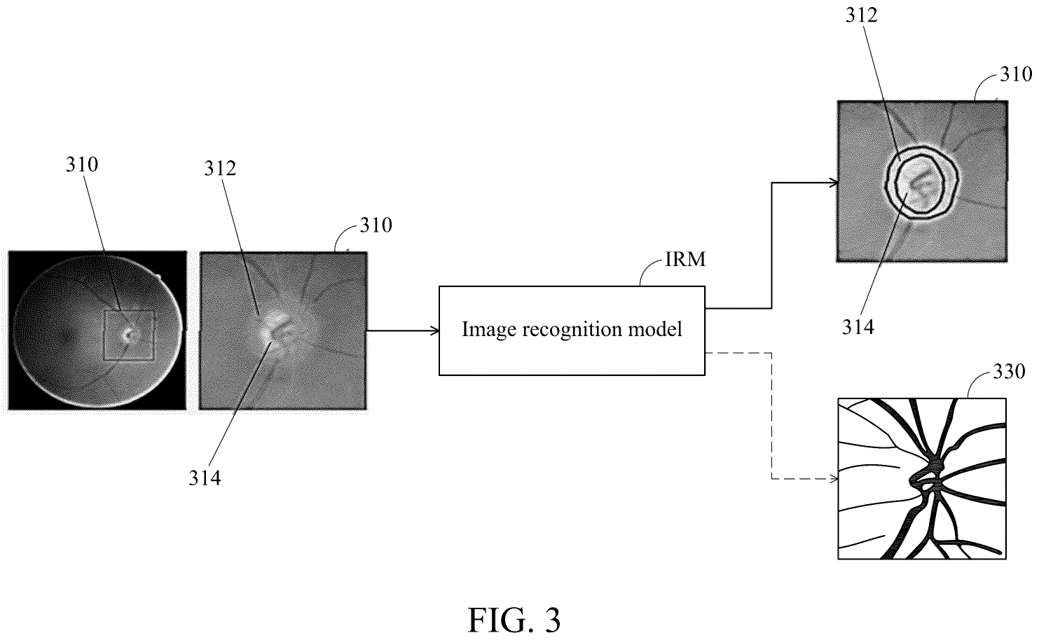

In one embodiment, the fundus image captured by the ophthalmoscope device 106, for example the fundus map 310 in FIG. 3, is transmitted to the image processing system 100, through wired or wireless transmission.

In addition, the image processing system 100 can be linked to a medical database (not shown FIG. 1) through a communication network such as the Internet or an Intranet to obtain the fundus map, for example the fundus map 310 in FIG. 3.

According to the present invention, the image processing method for analyzing an optic cup and an optic disc can be implemented by the processor 102 in the image processing system 100. The processor 102 reads out the programs and models stored in the storage device 104, and executes the programs and models.

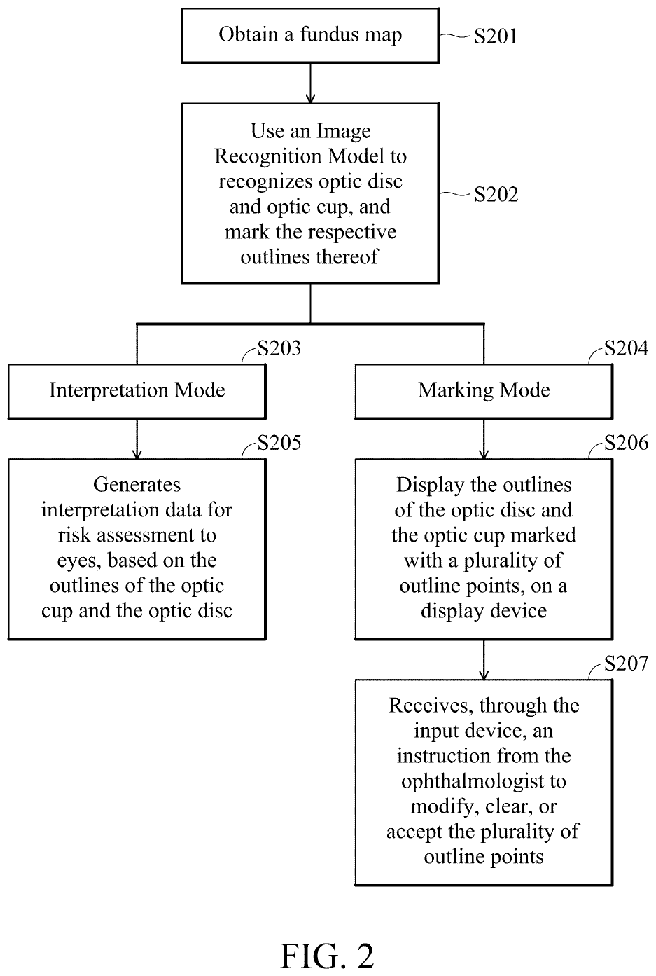

FIG. 2 shows a flow chart of an image processing method for analyzing an optic cup and an optic disc, according some exemplary embodiment of the present invention.

Referring to FIG. 2, in step S201, obtain a fundus map (for example, the fundus map 310 in FIG. 3) through using the processor 102 in the image processing system 100.

In one embodiment, the fundus map 301 will be an image of an eyeball. The eyeball image is generally red (or similar colors such as red and orange). The optic disc 314 and the optic cup 314 shows slightly different color areas (for example, yellow).

In step S202, the processor 102 recognizes an optic disc 312 and an optic cup 314 in the fundus map 310 (in view of FIG. 3), using an Image Recognition Model (IRM), also called image segmentation model. In addition, the image recognition model (IRM) further can mark the respective outlines of the optic disc 312 and the optic cup 314, as shown by the marking lines in FIG. 3.

In step S203, the processor 102 may operate in an interpretation mode. In addition, in step S204, the processor 102 may operate in a marking mode.

When the processor operates in the interpretation mode, the processor 102, in step S205, generates interpretation data for risk assessment of eyes, according to at least the outlines of the optic cup 314 and the optic disc 312.

In the marking mode, the processor 102, in step S206, displays the outlines of the optic disc 312 and the optic cup 314 marked with a plurality of outline points, on a display device (not shown in FIG. 1) of the image processing system 100 (for example, a notebook computer). Next, in step S207, the processor 102 receives an instruction from the ophthalmologist to modify, clear, or accept the plurality of outline points, through an input device (not shown in FIG. 1) of the image processing system 100.

In one embodiment, in the marking mode, the processor 102 further has a marking tool with a “pre-marking” outline function. It should be noted that the outlines of the optic disc 312 and the optic cup 314 marked by the aforementioned image recognition model IRM, called “pre-marked” outlines. The pre-marked outlines are recommended or temporary markings, which can be used by the ophthalmologist to observe the features of the optic disc and optic cup.

In one embodiment, operating the processor 102 in the marking mode can assist ophthalmologists in marking the optic disc and optic cup in the original fundus map. In this embodiment, the processor 102 obtains an original fundus map 310 in step S201. Next, the processor 102 further recognizes the optic disc 312 and the optic cup 314, using the image recognition model IRM, and generates the “pre-marked” outlines corresponding to the optic disc 312 and the optic cup 314. The processor 102 performs in the marking mode for the ophthalmologist to mark the optic cup and the optic disc, according to the following scenarios.

### Scenario (1):

The processor 102 displays the fundus map and the “pre-marked” outlines on the display device for review by the ophthalmologist. If the ophthalmologist completely accepts the “pre-marked” outlines, the processor 102 ends the marking of the optic cup and disc, based on the instructions input by the ophthalmologist through the input device.

### Scenario (2):

If the ophthalmologist only partially accepts the “pre-marked” outlines in the fundus map based on his professional and practical opinions, the processor 102 may further provide a modification tool so that the ophthalmologist can partially modify the “pre-marked” outlines through the input device. For example, after adjusting or changing the positions of the multiple outline points representing the “pre-marked” outlines, the marking of the optic cup and disc is finished by the processor 102.

### Scenario (3):

After inspecting the “pre-marked” outlines, the ophthalmologist may clear all outline points through the input device, and manually mark the outline of the optic disc or/and the optic cup.

Traditionally, when an ophthalmologist marks the optic cup and the optic disc, they must manually mark coordinate points one by one to sketch the outline, and it is a time-consuming task. However, according to this embodiment, since the processor 102 can perform in the marking mode, the marking process to modify or change the outline points (coordinate points) of the “pre-marked” outlines is simplified, thereby accelerating the marking process, such as in the aforementioned scenario (2), but it is not limited thereto.

In one embodiment of the present invention, the image recognition model IRM is a deep learning model that has been trained by using a large number of pre-collected fundus maps as training data. It should be noted that on each of the pre-collected fundus maps (the training data) the outlines of the optic cup and the optic disc have been pre-marked by the ophthalmologist. In this way, when the image recognition model IRM (that is, the aforementioned trained deep learning model) used in this embodiment receives the original fundus map, it recognizes the optic disc and the optic cup in the fundus map, according to the color variation of the optic disc and its surroundings, the color variation between the optic disc and the optic cup, and/or the turning variation of blood vessel in the optic disc and cup, and then marks the outlines of the optic disc and the optic cup.

U-Net is a Convolutional Neural Network (CNN) developed for biomedical image recognition. U-Net is based on a fully convolutional neural network and is structurally modified and expanded so that it can generate more accurate segmentation and recognition with fewer training images. In one embodiment, the image recognition model IRM is implemented by using the U-Net, for example, but it is not limited thereto.

In one embodiment of the present invention, the image recognition model IRM may also be trained for deep learning application, by a large number of fundus maps with pre-marked blood vessel distribution. After training, the image recognition model IRM can recognize and obtain blood vessel segmentation image form the original fundus map. In addition, the trained image recognition model IRM can be output to various application devices, such as the computer devices in hospital, notebook computers or handheld medical devices of ophthalmologists. In this embodiment, the trained image recognition model IRM is stored in the storage device 104 of the notebook computer (image processing device 100).

Therefore, after receiving the fundus map 310, the processor 102 can also obtain the blood vessel segmentation image form the fundus map 310. The processor 102 can further selectively generate a blood vessel segmentation map 330 corresponding to the fundus image 310 through the image recognition model IRM according to the application situation. In this way, the processor 102 can extract the shape and thickness of the blood vessels from the blood vessel segmentation map, and use it in conjunction with the color difference between the optic cup and the optic disc in the fundus map, to more accurately recognize the optic cup and the optic disc.

For example, the processor 102 can recognize and mark the optic disc based on the color difference between the optic disc and its surroundings in the fundus image through the image recognition model IRM; or based on the blood vessel turning, shape and thickness in the blood vessel segmentation map, and the aforementioned color difference in the fundus map, at the same time.

In one embodiment, in the interpretation mode, the processor 102 in step S205, generates the interpretation data based on the outline of the optic disc 312 and the outline of the optic cup 314, for example, based on the cup-to-optic ratio (CDR), Vertical Cup-to-Disc Ratio (VCDR), or Rim-to-Disc Ratio (RDR) of the optic cup to the optic disc, for ophthalmologists to diagnose eye injuries risk assessment and further provide suggestions to the inspection object.

Through the interpretation mode and the marking mode provided in the image processing system and method for analyzing optic cup and optic disc of the present invention, the task to mark the optic cup and disk outlines can be assisted, thereby reducing the ophthalmologist's time for marking, and at the same time helping to accelerate risk assessment process and improve interpretation quality.

Although the present invention has been disclosed in the above exemplary embodiments, it is not intended to limit the present invention. The person skilled in the art can make various changes and modifications without departing from the spirit and scope of the present invention. Therefore, the scope of the present invention shall be determined by the appended claims.

What is claimed is:

1. An image processing system for analyzing an optic cup and optic disc, comprising:
a processor, accessing a program to perform the following operations:
receiving a fundus map, and using an image recognition model to recognize an optic cup and an optic disc in the fundus map, and to mark outlines of the optic cup and the optic disc;
in an interpretation mode, generating interpretation data for risk assessment of eyes, according to at least the outlines of the optic cup and the optic disc;
wherein the image recognition model is a deep learning model which has been trained by using pre-collected fundus maps as training data, and
on each of the pre-collected fundus maps, the outlines of the optic cup and the optic disc have been previously marked by an ophthalmologist.

2. The image processing system for analyzing an optic cup and optic disc as claimed in claim 1, further comprising a display device, and an input device;
wherein the processor further performs the following operations:
in a marking mode, marking the outlines of the optic cup and the optic disc with a plurality of outline points,
displaying the plurality of outline points on the display device; and
through the input device, receiving instructions to modify, clear, or accept the plurality of outline points, from the ophthalmologist.

3. The image processing system for analyzing an optic cup and optic disc as claimed in claim 1, wherein
the image recognition model further recognizes a blood vessel segmentation map from the fundus map, and
the image recognition model recognizes the optic disc based on the color difference between the optic disc and the surroundings thereof, or based on the aforementioned color difference and the blood vessel segmentation map simultaneously.

4. The image processing system for analyzing an optic cup and optic disc as claimed in claim 1, wherein
the image recognition model further recognizes a blood vessel segmentation map corresponding to the fundus map, and
the image recognition model further recognizes the optic cup from the optic disc, according to the shapes and turns of blood vessels in the blood vessel segmentation map, and the color difference and shape difference compared with the optic disc.

5. The image processing system for analyzing an optic cup and optic disc as claimed in claim 1, wherein
the interpretation data is a Cup-to-Disc Ratio (CDR), or a Rim-to-Disc Ratio (RDR), which is obtained by the processor based on the outlines of the optic cup and the optic disc.

6. An image processing method for analyzing an optic cup and optic disc, applied to an electronic apparatus, the method comprising:
obtaining a fundus map through a processor of the electronic apparatus;
recognizing an optic cup and an optic disc in the fundus map, and marking outlines of the optic cup and the optic disc;
in an interpretation mode, using the processor to generate interpretation data for risk assessment of eyes according to at least the outlines of the optic cup and the optic disc;
wherein the image recognition model is a deep learning model which has been trained by using pre-collected fundus maps as training data, and
on each of the pre-collected fundus maps, the outlines of the optic cup and the optic disc have been previously marked by an ophthalmologist.

7. The image processing method for analyzing an optic cup and optic disc as claimed in claim 6, further comprising:
using the processor while in a marking mode to mark the outlines of the optic cup and the optic disc with a plurality of outline points,
displaying the plurality of outline points on a display device of the electronic apparatus; and
directing the processor to modify, clear, or accept the plurality of outline points, according to instructions from the ophthalmologist, the processor receiving the instructions through an input device of the electronic apparatus.

8. The image processing method for analyzing an optic cup and optic disc as claimed in claim 6, wherein
the image recognition model is used to further recognize a blood vessel segmentation map from the fundus map, and
the image recognition model is used to further recognize the optic disc, based on the color difference between the optic disc and the surroundings thereof, or based on the aforementioned color difference and the blood vessel segmentation map.

9. The image processing method for analyzing an optic cup and optic disc as claimed in claim 6, wherein
the image recognition model is used to further recognize a blood vessel cut map corresponding to the fundus map, and
the image recognition model is used to further recognize the optic cup in the optic disc, according to the shapes and turns of blood vessels in the blood vessel segmentation map and the color difference and shape difference from the optic disc.

10. The image processing method for analyzing an optic cup and optic disc as claimed in claim 6, wherein
the interpretation data is the Cup-to-Disc Ratio (CDR), or the Rim-to-Disc Ratio (RDR), which is obtained by the processor based on the outlines of the optic cup and the optic disc.
